(19) United States
(12) Patent Application Publication
(10) Pub. No.: US 2025/0099662 A1
(43) Pub. Date: Mar. 27, 2025

(54) PERITONEAL DIALYSATE FLOW PATH SENSING

(71) Applicant: Mozarc Medical US LLC, Minneapolis, MN (US)

(72) Inventors: Martin T. Gerber, Maple Grove, MN (US); Christopher M. Hobot, Rogers, MN (US)

(21) Appl. No.: 18/975,612

(22) Filed: Dec. 10, 2024

Related U.S. Application Data
(63) Continuation of application No. 17/187,256, filed on Feb. 26, 2021, now Pat. No. 12,194,214.
Application No. 17/187,256 is a continuation of application No. 15/666,614, filed on Aug. 2, 2017, now Pat. No. 10,994,064.
(60) Provisional application No. 62/373,133, filed on Aug. 10, 2016.

(51) Int. Cl. A61M 1/28; A61M 1/16

(52) U.S. Cl. CPC A61M 1/28; A61M 1/1609; A61M 1/1619; A61M 1/1696; A61M 2205/3317; A61M 2205/3324; A61M 2205/3327; A61M 2205/3331; A61M 2205/3337; A61M 2205/3368; A61M 2205/50; A61M 2205/502; A61M 2205/52; A61M 2205/702

(57) ABSTRACT

The invention relates to systems and methods for sensing fluid characteristics of peritoneal dialysate infused into and removed from a patient during treatment. The systems and methods include sensors, processors, and flow paths for determining patient health based on the fluid characteristics of the peritoneal dialysate. The system can be a peritoneal dialysis cycler which can include an infusion line; an effluent line; at least one pump positioned in the infusion and/or effluent line; and at least one sensor fluidly connected to the effluent line. The sensor can be at least one of a flow sensor, an ion selective electrode, a pH sensor, a pressure sensor, a refractive index sensor, and a temperature sensor. The method can include infusing peritoneal dialysate through an infusion line; removing peritoneal dialysate through an effluent line; and determining at least one fluid characteristic of the peritoneal dialysate in the effluent line.

### CROSS-REFERENCE TO RELATED APPLICATIONS

This application is a continuation of U.S. patent application Ser. No. 17/187,256, filed Feb. 26, 2021, which claims the benefit of and priority to U.S. patent application Ser. No. 15/666,614 filed Aug. 2, 2017, which claims priority to Provisional Patent Application No. 62/373,133 filed Aug. 10, 2016, the entire disclosures of each of which are incorporated by reference herein.

### FIELD OF THE INVENTION

The invention relates to systems and methods for sensing fluid characteristics of peritoneal dialysate infused into and removed from a patient during treatment. The systems and methods include sensors, processors, and flow paths for determining patient health based on the fluid characteristics of the peritoneal dialysate.

### BACKGROUND

Peritoneal Dialysis (PD) is a dialysis treatment that differs from Hemodialysis (HD) because blood is not removed from the body and passed through a dialyzer, but a catheter is placed in the peritoneal cavity and dialysate removed and introduced directly into the peritoneal cavity. Blood is cleaned inside the patient using the patient's own peritoneum as a type of dialysis membrane. The two primary classes of PD are Continuous Ambulatory Peritoneal Dialysis (CAPD) and Continuous Cycling Peritoneal Dialysis (CCPD) (or Automated Peritoneal Dialysis (APD)). In CAPD, dialysis is performed continuously by positioning a bag of peritoneal dialysate at shoulder level and using gravity to pull the fluid into the peritoneal cavity. The used dialysate is then drained from the cavity and discarded. The time period that the dialysate is in the cavity is called the dwell time and can range from 30 minutes to 4 hours or more. CAPD is typically performed three, four or five times in a 24-hour period while a patient is awake. CAPD requires no cycler to deliver and remove the fluid.

Determination of specific fluid characteristics of the peritoneal dialysate infused into and removed from the patient allows for optimization of treatment and early medical intervention in the event of worsening health. Known systems do not provide adequate mechanisms to determine fluid characteristics of the dialysate used in peritoneal dialysis during treatment. In particular, known systems do not allow the effluent or filtrate removed from the patient to be sensed.

Hence, there is a need for systems and methods for determining fluid characteristics of the peritoneal dialysate. The need extends to systems and methods monitoring the fluid characteristics during treatment. There is also a need for systems and methods to remove portions of the peritoneal dialysate over the course of treatment to determine any changes to the peritoneal dialysate while in the peritoneal cavity of the patient.

### SUMMARY OF THE INVENTION

The first aspect of the invention relates to a peritoneal dialysis cycler. In any embodiment, the peritoneal dialysis cycler can include a combined infusion and effluent line for delivering and receiving a peritoneal dialysate to and from a peritoneal cavity; at least one pump positioned in the combined infusion and effluent line; and at least one sensor fluidly connected to the combined infusion and effluent line, wherein the at least one sensor is selected from the group of: a flow sensor, an ion selective electrode, a pH sensor, a pressure sensor, a refractive index sensor, and a temperature sensor. In any embodiment, the combined infusion and effluent line can be separated into two different lines, which can be referred to as an infusion line and an effluent line.

In any embodiment, the peritoneal dialysis cycler can include a sampling flow path fluidly connected to the combined infusion and effluent line, wherein the at least one sensor is positioned in the sampling flow path; a valve connecting the combined infusion and effluent line to the sampling flow path; and at least one pump in the sampling flow path.

In any embodiment, the peritoneal dialysis cycler can include a detachable sampling reservoir fluidly connected to the sampling flow path.

In any embodiment, the peritoneal dialysis cycler can include a peritoneal dialysate generation flow path fluidly connected to the combined infusion and effluent line; the peritoneal dialysate generation flow path having a water source; a water purification module; at least one concentrate source fluidly connected to the peritoneal dialysate generation flow path; and at least one sensor positioned in the peritoneal dialysate generation flow path or infusion line. If two separate infusion and effluent lines are used, the peritoneal dialysate generation flow path can be fluidly connected to the infusion line.

In any embodiment, the peritoneal dialysis cycler can include a dialysate regeneration module; the dialysate regeneration module fluidly connected to the combined infusion and effluent line and the peritoneal dialysate generation flow path. If two separate infusion and effluent lines are used, the dialysate regeneration module can be fluidly connected to the effluent line.

In any embodiment, the dialysate regeneration module can be positioned downstream of the sampling flow path.

In any embodiment, the peritoneal dialysis cycler can include a processor in communication with the at least one sensor; the processor receiving data from the sensor and storing the data in a machine-readable storage medium.

In any embodiment, the processor can include an input/output interface, the input/output interface providing data from the at least one sensor to a user.

In any embodiment, the peritoneal dialysis cycler can include a sampling port fluidly connected to the combined infusion and effluent line. If two separate infusion and effluent lines are used, the sampling port fluidly connected can be fluidly connected to the effluent line.

In any embodiment, the sampling port can be covered by a pierceable septum.

The features disclosed as being part of the first aspect of the invention can be in the first aspect of the invention, either alone or in combination.

The second aspect of the invention is drawn a method. In any embodiment, the method can include the steps of infusing peritoneal dialysate into a patient through a combined infusion and effluent line; and determining at least one fluid characteristic of the peritoneal dialysate in the effluent line. In any embodiment, the method can include the steps of infusing peritoneal dialysate into a patient through a separate infusion line; removing peritoneal dialysate from the patient through a separate effluent line.

In any embodiment, the method can include the step of pumping the peritoneal dialysate from the effluent line to a sampling reservoir; wherein the step of determining at least one fluid characteristic of the peritoneal dialysate includes determining the fluid characteristic in the peritoneal dialysate in the sampling reservoir.

In any embodiment, the method can include the step of adding one or more reagents to the peritoneal dialysate in the sampling reservoir prior to determining the fluid characteristic.

In any embodiment, the method can include the step of removing a portion of fluid from the combined infusion and effluent line through a sampling port, wherein the step of determining at least one fluid characteristic of the peritoneal dialysate includes determining the fluid characteristic in the removed fluid. If two separate infusion and effluent lines are used, the step of removing a portion of fluid can be performed using the effluent line.

In any embodiment, the method can include the step of determining at least one fluid characteristic in the peritoneal dialysate in the combined infusion and effluent line. If two separate infusion and effluent lines are used, the step of determining at least one fluid characteristic in the peritoneal dialysate can be performed using the infusion line.

In any embodiment, at least one fluid characteristic can be determined in any one of the combined infusion and effluent line, the infusion line, and the effluent line.

In any embodiment, the fluid characteristic can be selected from the group of a pH of the fluid, and a volume of the fluid.

In any embodiment, the method can include the steps of a portion of the peritoneal dialysate from the patient through the effluent line at a first time; removing a portion of the peritoneal dialysate from the patient through the effluent at a second time; and determining the fluid characteristic at the first time and the second time.

In any embodiment, the fluid characteristic can be selected from the group of pH and concentration of one or more solutes.

In any embodiment, the method can include the step of communicating the fluid characteristic to a machine-readable storage medium in a processor.

The features disclosed as being part of the second aspect of the invention can be in the second aspect of the invention, either alone or in combination.

### DESCRIPTION

### Brief Description of the Drawings

FIG. 1 is a schematic of a system for sensing fluid characteristics of peritoneal dialysate in a flow path.

FIG. 2 is a schematic of a regenerative peritoneal dialysis system for sensing fluid characteristics of the peritoneal dialysate.

FIG. 3 is a flow chart illustrating a method of determining fluid characteristics in a sample of peritoneal dialysate.

### DETAILED DESCRIPTION OF THE INVENTION

Unless defined otherwise, all technical and scientific terms used generally have the same meaning as commonly understood by one of ordinary skill in the art.

The articles “a” and “an” are used to refer to one or to over one (i.e., to at least one) of the grammatical object of the article. For example, “an element” means one element or over one element.

The term “combined infusion and effluent line” refers to a fluid connector for delivering and removing fluid from a peritoneal cavity of a patient. The combined infusion and effluent line can optionally be separated into an independent infusion line and an independent effluent line.

The terms “communication” and “communicating” refer to an electronic or wireless link between two components.

The term “comprising” includes, but is not limited to, whatever follows the word “comprising.” Use of the term indicates the listed elements are required or mandatory but that other elements are optional and can be present.

A “concentrate source” is a source of one or more solutes. The concentrate source can have one or more solutes with a solute concentration greater than the solute concentration to be used for dialysis. The concentrate in the concentrate source can also be lower than the solute concentration generally used in dialysis for generation of low concentration dialysate.

The terms “concentration” and “solute concentration” refers to an amount of a solute dissolved in a given amount of a solvent.

The term “conductivity sensor” refers to any component capable of measuring the electrical conductance or the electrical resistance of a fluid.

The term “consisting of” includes and is limited to whatever follows the phrase “consisting of.” The phrase indicates the limited elements are required or mandatory and that no other elements can be present.

The term “consisting essentially of includes whatever follows the term” consisting essentially of and additional elements, structures, acts or features that do not affect the basic operation of the apparatus, structure or method.

The term “detachable” relates to any component of that can be separated from a system, module, cartridge or any component of the invention. “Detachable” can also refer to a component that can be taken out of a larger system with minimal time or effort. In certain instances, the components can be detached with minimal time or effort, but in other instances can require additional effort. The detached component can be optionally reattached to the system, module, cartridge or other component.

The terms “determining” and “determine” refer to ascertaining a particular state of a system or variable(s).

The term “dialysate regeneration module” refers to a component or components capable of removing waste products from a fluid.

The term “downstream” refers to a position of a first component in a flow path relative to a second component wherein fluid will pass by the second component prior to the first component during normal operation. The first component can be said to be “downstream” of the second component, while the second component is “upstream” of the first component.

The term “effluent line” refers to a fluid connector for removing fluid from a peritoneal cavity of a patient. The term “effluent line” can also refer to a combined effluent and infusion line.

The term “flow sensor” refers to any component capable of measuring a volume or a rate of fluid moving through a conduit.

A “fluid” is a liquid substance optionally having a combination of gas and liquid phases in the fluid. Notably, a liquid can therefore also have a mixture of gas and liquid phases of matter.

A “fluid characteristic” is any sensed characteristic of a fluid, including temperature, pressure, concentration, color, or any other characteristic.

The terms “fluidly connectable,” “fluidly connected,” “fluid connection” “fluidly connectable,” or “fluidly connected” refer to the ability to pass fluid, gas, or mixtures thereof from one point to another point. The two points can be within or between any one or more of compartments, modules, systems, and components, all of any type.

The term “infusing” or to “infuse” a fluid refers to the movement of peritoneal dialysate into the peritoneal cavity of a patient.

An “infusion line” is a fluid line for carrying peritoneal dialysate into a body cavity or part of a patient such as a peritoneal cavity. The term “infusion line” can also refer to a combined effluent and infusion line.

The term “input/output interface” refers to a module of a processor or computing system that allows data to be received by the processor or computing system and provided by the processor or computing system. The input/output interfaces can automatically receive and provide data from sensors, or can receive data manually input through the interface, such as by a keyboard.

An “integrated cycler” is a component for movement of fluid into and out of the peritoneal cavity of a patient, wherein the integrated cycler forms a part of an overall system. For example, the integrated cycler can be contained in a housing with other components used for peritoneal dialysis and be in fluid and electrical connection with desired components.

The term “ion selective electrode” refers to any component capable of determining a concentration of a specific ion in a fluid based on a detected electrical potential.

The term “machine-readable storage medium” refers to any electronic device capable of storing information in a digital format for reading by a computer, processor, or other electronic device.

A “patient” or “subject” is a member of any animal species, preferably a mammalian species, optionally a human. The subject can be an apparently healthy individual, an individual suffering from a disease, or an individual being treated for a disease.

“Peritoneal dialysate” is a dialysis solution to be used in peritoneal dialysis having specified parameters for purity and sterility. Peritoneal dialysate is different than a dialysate used in hemodialysis, although peritoneal dialysate can be used in hemodialysis.

A “peritoneal dialysate generation flow path” is a path used in generating dialysate suitable for peritoneal dialysis.

“Peritoneal dialysis” is a therapy wherein a dialysate is infused into the peritoneal cavity, which serves as a natural dialyzer. In general, waste components diffuse from a patient's bloodstream across a peritoneal membrane into the dialysis solution via a concentration gradient. In general, excess fluid in the form of plasma water flows from a patient's bloodstream across a peritoneal membrane into the dialysis solution via an osmotic gradient. Once the infused peritoneal dialysis solution has captured sufficient amounts of the waste components the fluid is removed. The cycle can be repeated for several cycles each day or as needed.

The term “peritoneal dialysis cycler” or “cycler” refers to components for movement of fluid into and out of the peritoneal cavity of a patient, with or without additional components for generating peritoneal dialysate or performing additional functions.

The term “pH” refers to the hydrogen ion concentration in a fluid.

The term “pH sensor” refers to any component capable of measuring the hydrogen ion concentration in a fluid.

The term “pierceable septum” refers to a component through which a needle or syringe can be inserted to draw fluid out of a flow path.

The term “portion of fluid” refers to an amount of a fluid less than the entire amount of the fluid in a flow path, container, or reservoir.

The term “positioned” refers to the location of a component.

The term “pressure sensor” refers to any component capable of determining the force exerted by a fluid.

The term “processor” is a broad term and is to be given its ordinary and customary meaning to a person of ordinary skill in the art. The term refers without limitation to a computer system, state machine, and/or processor designed to perform arithmetic or logic operations using logic circuitry that responds to and processes the basic instructions that drive a computer. In any embodiment of the first, second, third, and fourth invention, the terms can include ROM (“read-only memory”) and/or RAM (“random-access memory”) associated therewith.

The term “pump” refers to any device that causes the movement of fluids or gases by applying suction or pressure.

The terms “pumping,” “pumped,” or to “pump” refers to moving or flowing a fluid using a pump of any type known to those of ordinary skill in the art.

The term “reagent” refers to a substance that will react with a second substance to produce an observable change in a solution.

The term “receiving” or to “receive” means to obtain information from any source.

A “refractive index sensor” is any component capable of detecting the ratio of the speed of light through a fluid to the speed of light through water. The concentration of one or more solutes in the fluid can be determined based on the refractive index.

The term “removing” fluid refers to flowing fluid out of a container, system, or patient.

The term “sampling flow path” refers to a flow path diverted from a main flow path in which fluid characteristics of a fluid in the sampling flow path can be determined.

The term “sampling port” refers to a fluid port in a flow path through which a portion of the fluid in the flow path can be removed for analysis.

The term “sampling reservoir” refers to a container for collecting a portion of a fluid for analysis of the fluid separate from the rest of a system.

A “sensor” is a component capable of determining one or more states of one or more variables in a system.

A “solute” is a substance dissolved in, or intended to be dissolved in, a solvent.

The term “storing” or to “store” refers to saving electronic data or information in a machine readable medium.

A “temperature sensor” is any component capable of measuring the temperature of a fluid.

The term “transmitting” or to “transmit” refers to sending information electronically.

A “valve” is a device capable of directing the flow of fluid or gas by opening, closing or obstructing one or more pathways to allow the fluid or gas to travel in a path. One or more valves configured to accomplish a desired flow can be configured into a “valve assembly.”

The term “volume” refers to an amount of a fluid.

The term “water purification module” refers to a component or components capable of removing biological or chemical contaminants from water.

The term “water source” refers to a source from which potable water can be obtained.

### Peritoneal Dialysis Fluid Path Sensing

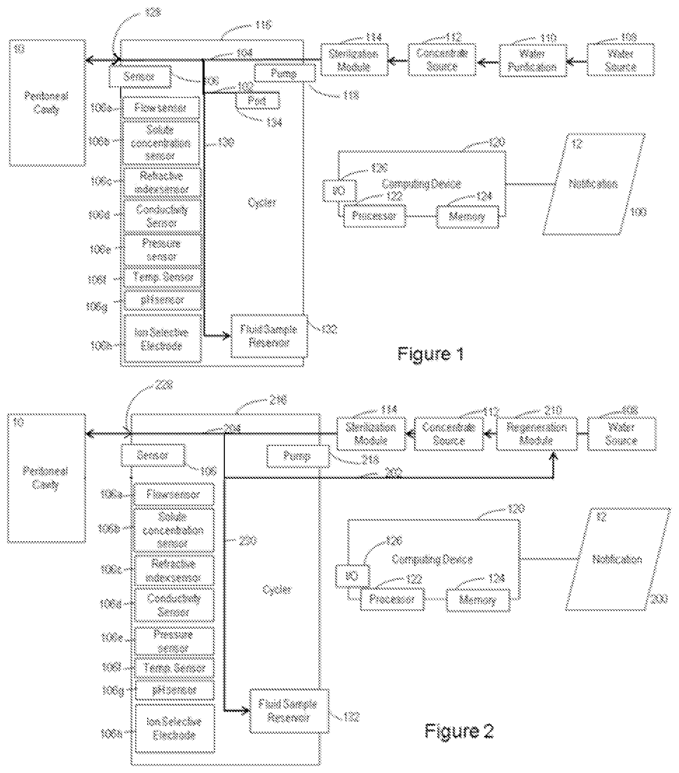

FIG. 1 illustrates a system 100 for sampling peritoneal dialysate removed from a peritoneal cavity 10 of a patient. The system 100 can include a combined peritoneal dialysate effluent line and infusion line 128, referred to herein as an effluent line, a peritoneal dialysate generation flow path 104, at least one sensor 106-106h, a peritoneal dialysis cycler 116, and a computing device 120. The effluent line 128 in the catheter has a single channel used for both filling and removal of effluent. One of skill in the art will understand that separate effluent and infusion lines can be used. The system 100 can be embodied an integrated cycler wherein the peritoneal dialysis cycler 116 includes the peritoneal dialysate effluent line 128, the peritoneal dialysate generation flow path 104, and the at least one sensor 106-106h forming the system 100 for sampling peritoneal dialysate removed from a peritoneal cavity 10. Alternatively, the peritoneal dialysis cycler 116 can be nonintegrated without the peritoneal dialysate generation flow path 104. Peritoneal dialysate can be prepared off-line and provided to the cycler 116. The computing device 120 can be a part of the peritoneal dialysis cycler 116, whether integrated or nonintegrated, or can be separate device in communication with the sensors.

The peritoneal dialysate effluent line 128 can be fluidly connected to a waste reservoir (not shown) to collect effluent. Optionally, a sampling flow path 130 can be in fluid communication with the peritoneal dialysate effluent line 102 for analysis of the fluid A valve (not shown) in the cycler can divert fluid from the effluent line 128 to the sampling flow path 130. The system 100 can divert a sample of effluent flowing through the peritoneal dialysate effluent line 102 to allow determination of fluid characteristics outside of the cycler 116. The sample can be diverted continuously or at specific intervals and in predetermined amounts. A valve (not shown) can be included to selectively divert peritoneal dialysate from the peritoneal dialysate effluent line 102 into the sampling flow path 130. A pump (not shown) can provide an additional driving force for moving peritoneal dialysate through the sampling flow path 130. A similar analysis can be conducted on the generated peritoneal dialysate by diverting a volume of generated peritoneal dialysate into the sampling flow path 130. Analysis of the generated peritoneal dialysate can serve as a quality check on the newly generated peritoneal dialysate, as well as calibration of the sensors by comparing sensed values to known values of the dialysate. Analysis of the newly generated dialysate can also be used by the system for self-learning or machine learning to adjust the dialysate composition to a precision beyond the capabilities of known systems. Analysis of the generated peritoneal dialysate can also be used as a safety system to ensure the concentration of solutes in the peritoneal dialysate is within a predetermined threshold of the expected values.

Alternatively or additionally, the system 100 can include a sampling port 134. The sampling port 134 can be fluidly connected to the peritoneal dialysate effluent line 102. The sampling port 134 can alternatively be fluidly connected to the sampling flow path 130. The sampling port 134 can be covered by a pierceable septum. A user can insert a needle or syringe through the pierceable septum to draw out a portion of the peritoneal dialysate in the effluent line 102 or sampling flow path 130. The pierceable septum can re-seal after removal of the needle or syringe to avoid contamination of the peritoneal dialysate.

When used with an integrated cycler, the peritoneal dialysate generation flow path 104 can include a water source 108, one or more water purification modules 110, a concentrate source 112, a sterilization module 114, and the peritoneal dialysis cycler 116. The concentrate source 112 can contain one or more solutes. The water source 108, water purification module 110, concentrate source 112, sterilization module 114, and peritoneal dialysis cycler 116 can be fluidly connectable to the peritoneal dialysate generation flow path 104. The peritoneal dialysate generation flow path 104 can be fluidly connected to the combined infusion and effluent line 128 to infuse peritoneal dialysate into the peritoneal cavity 10. One of skill in the art will understand that with a single concentrate source, solutes can be altered in the dialysate without changing the relative proportions of each solute. With multiple concentrate sources, each individual solute can be adjusted independently of all other solutes. The concentration of the ionic compounds in the ion concentrate source can also be lower than the concentration generally used in dialysis for generation of low concentration dialysate. Any number of concentrate sources and concentrate pumps can be used. A separate osmotic agent source and ion concentrate source can be used to adjust the osmotic agent concentration and other solute concentrations independently. Any solute usable in peritoneal dialysis can be included in the concentrate sources. The water source 108 can be a non-purified water source, such as tap water, wherein the water from the water source 108 can be purified by the system. A non-purified water source can provide water without additional purification, such as tap water from a municipal water source, water that has undergone some level of purification, but does not meet the definition of “purified water” provided, such as bottled water or filtered water. The water source can contain water meeting the WHO drinkable water standards provided in Guidelines for Drinking Water Quality, World Health Organization, Geneva, Switzerland, 4th edition, 2011. Alternatively, the water source 108 can be a source of purified water, meaning water that meets the applicable standards for use in peritoneal dialysis without additional purification. The system pumps water from the water source to a water purification module to remove chemical contaminants in the fluid in preparation of the dialysate. The water purification module can be a sorbent cartridge containing anion and cation exchange resins and/or activated carbon. The system can pump the fluid to a sterilization module 114 for sterilizing the peritoneal dialysate prior to infusion into the patient. The sterilization module 114 can include one or more of a first ultrafilter, a second ultrafilter, and a UV light source, or any combination thereof. The sterilization module can be any component or set of components capable of sterilizing the peritoneal dialysate.

The concentrate sources 112 can contain one or more solutes for generating the peritoneal dialysate from purified water. The concentrates in the concentrate source 112 are utilized to generate a peritoneal dialysis fluid that matches a dialysis prescription. A concentrate pump (not shown) in communication with the processor or computing unit controls the movement of fluid from the concentrate sources 112 into the peritoneal dialysate generation flow path 104. One of skill in the art will understand that any number of concentrate sources can be used, each containing concentrates of one or more substances. For example, the concentrate sources 112 can include any number of concentrates combined or in separate concentrate sources. One or more osmotic agent sources can be included in addition to a single ion concentrate source. Alternatively, multiple ion concentrate sources can be used with each ion concentrate in a separate concentrate source. Any combination of concentrates in any number of concentrate sources can be used with the invention. The concentrate sources can infuse each particular concentrate to provide an infused ion concentration that is lower than a prescribed amount for a particular patient. One desired outcome can be to provide a concentration for a particular ion that is lower than a patient's pre-dialysis ion concentration. Additionally, if multiple ion sources are to be delivered by a concentrate source, the present system can selectively dilute a desired ion while maintaining concentration levels for other ions. Hence, the present invention can avoid adjusting down every ion insofar as an added diluent may adversely affect concentrations of ions already in a normal range.

One or more fluid characteristics in the peritoneal dialysate removed from the patient can be determined by one or more sensors 106-106h. The sensors 106-106h can be fluidly connected to one or more of the peritoneal dialysate effluent line 128, the sampling flow path 130, the sampling reservoir 132, and the peritoneal dialysate generation flow path 104. For use with non-invasive sensors, the sensors 106 can be positioned in the effluent line 128 and the sampling flow path 130 can be optional. The sample can be tested while in the peritoneal dialysate effluent line 128, the peritoneal dialysate generation flow path 104, or in the sampling flow path 130. Additionally or alternatively, a sample can be diverted away from the peritoneal dialysate effluent line 102 and then tested. For example, the sample can be diverted into the sampling flow path 130 fluidly connected to the peritoneal dialysate effluent line 102. The sample can be diverted through the sampling flow path 130 into a detachable sampling reservoir 132 fluidly connected to the sampling flow path 130 for removal of the dialysate from the cycler 116 and off-line testing. Certain fluid characteristics, such as color or clarity of the dialysate can require specialized equipment not included in the effluent line 128, or the sampling flow path 130. The detachable sampling reservoir 132 allows a portion of the peritoneal dialysate to be removed for determining any fluid characteristics with sensors not present in the effluent line 128 or sampling flow path 130. The sample can be tested while in the sampling flow path 130 and/or the detachable sampling reservoir 132. Alternatively, the sample can be diverted directly to standalone system, such as a blood analyzer for analysis. Blood analyzers can determine several fluid characteristics, which can be included in the system. One non-limiting example of a standalone analyzer is the Stat Profile® Critical Care Xpress analyzer by Nova Biomedical, however any analyzer can be used. The standalone analyzer can be in communication with the processor or computing unit of the system to provide the system with the results of the analysis. Specialized tubing with a T-junction or a valve can be used to divert a volume of fluid into the sampling flow path or to a standalone analyzer. In an embodiment, one or more sensors 106-106h can be external to the peritoneal dialysis cycler 116 and the sample can be tested external to the peritoneal dialysis cycler 116 in the sampling flow path 130. The system an also include duplication of analysis with duplicated sensors in multiple locations. For example, the same type of sensor can be included in both the effluent line 128 and in the sampling flow path 130. Alternatively, a separate analyzer can be included for duplication of analysis. Duplication of the analysis allows calibration of the sensors and acts as a safety check to ensure the sensors are properly functioning. The duplicated sensors can be attached to the cycler 116 or in a standalone system.

The one or more sensors can be separate sensors or one or more combined sensors. The one or more sensors 106-106h can be in fluid communication with and positioned in or along one or more of the peritoneal dialysate effluent line 128, the sampling flow path 130, the detachable sampling reservoir 132, and the peritoneal dialysate generation flow path 104.

Multiple instances of the one or more sensors 106-106h are shown in FIG. 1. For example, a flow sensor 106a can measure a volume of peritoneal dialysate removed from a patient. A solute concentration sensor 106b can measure a solute concentration of the peritoneal dialysate removed from the patient. The solute concentration sensor 106b can include a conductivity sensor or an ion selective electrode for determining the concentration of the ionic components of peritoneal dialysate removed from the patient. A refractive index sensor 106c can measure glucose or other osmotic agent concentration in the peritoneal dialysate removed from the patient. A conductivity sensor 106d can measure conductivity of the peritoneal dialysate removed from the patient. A pressure sensor 106e can measure a pressure of peritoneal dialysate removed from the patient, and/or a pressure to infuse peritoneal dialysate into the patient, when included in infusion line 104. A temperature sensor 106f can measure a temperature of peritoneal dialysate removed from the patient. A pH sensor 106g can measure a pH level of peritoneal dialysate removed from the patient. An ion selective electrode 106h can measure the concentration of one or more specific solutes in the peritoneal dialysate removed from the patient. Table 1 provides non-limiting examples of sensors and methods that can determine solute concentrations for a variety of solutes. Any one or more of the reagents in Table 1 can be added to the peritoneal dialysate to determine a fluid characteristic. One of skill in the art will understand that alternative or additional methods can be used, and any sensor or method known in the art can be incorporated.

TABLE-US-00001 TABLE 1 Analyte Test Name Key Reagents Detection Total A280 None UV/Visible protein spectrophotometer @ 280 nm Total Coomassie (Bradford Coomassie Brilliant Blue UV/Visible protein Assay) G-250 spectrophotometer @ 595 nm Total Bicinchoninic Acid Bicinchoninic acid UV/Visible protein (BCA, Smith Assay) Copper (II) sulfate spectrophotometer @ Lowry Assay 562 nm Total Pierce assay Proprietary Dye UV/Visible protein compounds spectrophotometer @ 660 nm Calcium O-cresolphthalein UV/Visible spectrophotometer @ 575 nm Calcium None Ion selective electrode Potassium None Ion selective electrode Magnesium None Ion selective electrode Glucose Glucose oxidase+Potentiometric platinum electrode that reduces hydrogen peroxide to produce an electric signal Glucose Glucose oxidase+UV/Visible peroxide reactive dye spectrophotometer @ (several available) dye specific wavelength

Referring to the tests listed in Table 1, UV/Vis spectrophotometry is an absorption spectroscopy or reflectance spectroscopy technique that operates in the visible or ultraviolet spectral range. A UV/Vis spectrophotometer exposes a chemical sample to light at predetermined wavelength and measures either the absorption or reflection spectra that is produced. The absorbance of the solution is proportional to the concentration of the absorbing species and the path length, so the concentration of the unknown sample can be quantified using a calibration curve developed using a series of samples of known concentration. Determination of protein concentration by measuring absorbance at 280 nm (A280) is based on the absorbance of UV light by the aromatic amino acids tryptophan and tyrosine, and by cystine, disulfide bonded cysteine residues, in protein solutions. Absorption correlates with concentration, which can be quantified using a calibration curve developed with standards of known concentration. The Bradford (Coomassie) assay reacts Coomassie blue dye with protein in an acidic/methanol solution. The protein-dye complex has a blue color, whereas the unbound dye has a brown color. The amount of protein in the solution can be quantified by measuring the intensity of the blue color at 595 nm and comparing to a calibration curve developed with standards of known concentration. The BCA (Smith) Assay primarily relies on two reactions. First, the peptide bonds in protein reduce Cu.sup.2+ ions from the copper (II) sulfate to Cu.sup.+ (a temperature dependent reaction). The amount of Cu.sup.2+ reduced is proportional to the amount of protein present in the solution. Next, two molecules of bicinchoninic acid chelate with each Cu.sup.+ion, forming a purple-colored complex that strongly absorbs light at a wavelength of 562 nm. Other commercially available protein assays have been developed to provide greater specificity and/or address interferences that can decrease utility of the assays described above. Many of the assays use proprietary dye molecules, but all use the general procedure of preparing a protein-dye complex that results in a color change that can be detected spectrophotometrically. Calcium ions (Ca2+) react with o-cresolphthalein complexone in an alkaline solution to form an intense violet colored complex which maximally absorbs at 577 nm. 2,3 8-Hydroxyquinoline can be added to remove interference by magnesium and iron. In the method the absorbance of the Ca-oCPC complex is measured bichromatically at 570/660 nm. The resulting increase in absorbance of the reaction mixture is directly proportional to the calcium concentration in the sample. An ion-selective electrode (ISE) is a transducer (or sensor) that converts the activity of a specific ion dissolved in a solution into an electrical potential. The voltage is theoretically dependent on the logarithm of the ionic activity, according to the Nernst equation. The ISE has a coating over the electrodes that allow specific ions to interact with the electrodes, but reject other ions. Many types of ISE are commercially available with different specificity and durability as needed for a specific application. ISE electrodes are available for calcium, magnesium, potassium and other ions of interest in PD fluid. Glucose can be quantified using a sensor that utilizes glucose oxidase. Glucose oxidase is an enzyme that oxidizes glucose to D-glucono-1,5 lactone+hydrogen peroxide. The hydrogen peroxide that is produced can be reduced on a platinum electrode to produce an electrical signal proportional to concentration. Alternatively, the peroxide can be complexed with a reactive dye, such as Amplex® Red reagent (10-acetyl-3,7-dihydroxyphenoxazine) to produce a colored complex that can be quantified using a UV/Vis spectrophotometer. Other peroxide reactive dyes are commercially available to measure peroxide concentration.

The computing device 120 can include the one or more processors 122, memory 124, and one or more input/output interfaces 126. One of ordinary skill in the art will recognize that the memory 124 can include long-term memory and operating memory, and/or memory serving as both long-term memory and operating memory. The memory 124 can be a machine-readable storage medium. The memory 124 can be in communication with the processor 122 and store instructions that when executed perform any of the methods of the present invention. The input/output interface(s) 126 can include an input port to receive information from the one or more sensors 106-106h, and an output interface to output data to a user, such as a notification regarding the sample. The processor 122 can be in communication with the at least one sensor 106-106h and store data received from the at least one sensor 106-106h in the memory 124. As with all features of the present application, intervening components, such as the input/output interface 126, can be present between the processor 122 and the sensors 106-106h. The computing device 120 can be a stand-alone device independent of the peritoneal dialysis cycler 116, or can be a part of the peritoneal dialysis cycler 116. The computing device 120 can be a remote device in network communication with the sensor(s) 106-106h, such as via the Internet.

FIG. 2 shows an alternative system 200 for sampling peritoneal dialysate to determine one or more fluid characteristics of the dialysate removed from the patient. A difference between system 200 and system 100 is the provision of a peritoneal dialysate regeneration module 210. A discussion of some features similar to the features of system 100 is omitted in the interest of brevity.

The peritoneal dialysis cycler 216 can include a pump 218, a combined infusion and effluent line 228, referred to herein as an effluent line, and a dialysate regeneration line 202. The effluent line 228 can be fluidly connected to the peritoneal dialysate generation flow path 204 downstream of the sterilization module 114. The peritoneal dialysate regeneration line 202 can be fluidly connected to the peritoneal dialysate generation flow path 204 upstream of the peritoneal dialysate regeneration module 210. The peritoneal dialysate regeneration module 210 can be positioned downstream of the optional sampling flow path 230.

Certain fluid characteristics require additional reagents or dyes for determination. In one non-limiting example, determination of glucose concentration requires that glucose react with glucose oxidase to produce hydrogen peroxide. The hydrogen peroxide formed through the reaction together with 4 amino-antipyrene (4-AAP) and phenol in the presence of peroxidase yield a red quinoeimine dye that can be measured spectrophotometrically at 505 nm. Alternatively, the hydrogen peroxide can be reacted with an appropriate electrode sensor that produces electric current in proportion to glucose concentration. Similarly, the protein content in peritoneal dialysate effluent can be detected by any suitable protein bioassay. In one non-limiting example, Coomassie Brilliant Blue G-250 dye is reacted with protein to form a colored complex that can be detected spectrophotometrically. The color intensity correlates with protein concentration. One of skill in the art will understand that alternative reagents can be used to determine the same or different fluid characteristics. Many of the reagents cannot be passed back to the patient when the peritoneal dialysate is regenerated and reused. The sampling flow path 230 allows necessary reagents to be added to the dialysate removed from the patient in a diverted flow path, ensuring that the reagents are not passed back into the dialysate generation flow path 204 and to the patient. Sensors that do not require the addition of reagents can alternatively be present in the effluent line 128, and the sampling flow path 130 is optional. A detachable sampling reservoir can allow a portion of the peritoneal dialysate removed from the patient to be analyzed off-line.

The system 200 can include a peritoneal dialysate effluent line 228, a peritoneal dialysate generation flow path 204, at least one sensor 106-106h, a peritoneal dialysis cycler 216, and a computing device 120 as shown in FIG. 1.

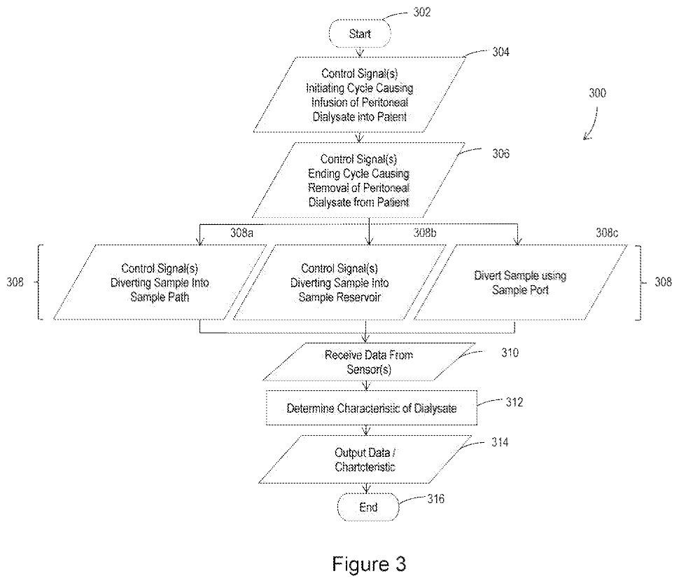

The operation of the systems 100, 200 of FIGS. 1 and 2 is shown in FIG. 3, which is a schematic representation of an exemplary computerized method 300 for sampling peritoneal dialysate removed from a peritoneal cavity 10 of a patient. In operation 302, the method 300 can start. A peritoneal dialysis session can be initiating or already underway in operation 302.

In operation 304, a control signal(s) initiating a cycle of the peritoneal dialysis session can be issued by the processor 122 of the system 100, 200 controlling components of peritoneal dialysate generation flow path 104, 204. Peritoneal dialysate can be infused into the peritoneal cavity 10 of a patient through the effluent line 128, 228. The method can proceed to operation 306.

In operation 306, a control signal(s) ending the cycle of the peritoneal dialysis session can be issued by the processor 122 of the system 100, 200. The control signal can cause the peritoneal dialysis cycler 116, 216 to initiate a drain portion of the cycle. Peritoneal dialysate can be removed from the peritoneal cavity of the patient 10 through the peritoneal dialysate effluent line 128, 228. The method can proceed to operation 308.

In operation 308, a control signal(s) diverting a sample of the peritoneal dialysate flowing through the peritoneal dialysate effluent line 128, 228 can be issued by the processor 122. Multiple instances of operation 308 are depicted in FIG. 3. For example, in operation 308a, the control signal(s) can cause the sample to be removed from the peritoneal dialysate flowing through the peritoneal dialysate effluent line 128, 228 and into the sampling flow path 130, 230. In operation 308b, the control signal(s) can cause the sample to be removed from the peritoneal dialysate flowing through the peritoneal dialysate effluent line 128, 228 and into the detachable sampling reservoir 132. In any embodiment, the sample can be pumped from the effluent line 128, 228, through a valve into the sampling flow path 130 and optionally to the detachable sampling reservoir 132. Alternatively, the sample can be diverted from the peritoneal dialysate effluent line 128, 228 and directly into the detachable sampling reservoir 132 or a standalone analyzer. In operation 308c, the peritoneal dialysate can be removed from the effluent line 128, 228 through a sampling port. For example, a syringe needle can be mechanically or manually inserted through the pierceable septum and a portion of the peritoneal dialysate can be removed for off-line analysis. Alternatively, one or more of the sensors can be positioned in the effluent line 128, 228, and the system need not divert a portion of the effluent. The method 300 can proceed to operation 310.

In operation 310, data can be received by the processor 122 from the sensors 106-106h regarding the sample. For example, the one or more sensors in fluid communication with the sampling flow path 130 can receive data representing a characteristic of the peritoneal dialysate. Alternatively, one or more sensors in the peritoneal dialysate effluent line 128, 228, the peritoneal dialysate generation flow path 104, 204, or a standalone analyzer, can receive data representing a characteristic of the peritoneal dialysate. Additionally or alternatively, the data received in operation 310 can be analyzed by the processor 122 to determine the characteristic of the peritoneal dialysate in operation 312. The data received from the sensors in operation 310 can be stored in a machine-readable storage medium. The method 300 can proceed to operation 314, and the data received from the sensors can be transmitted to a user. As another example, the one or more sensors in fluid communication with the detachable fluid reservoir 132 can receive data representing a characteristic of the peritoneal dialysate in the peritoneal dialysate effluent line 128, 228. As yet another example, one or more sensors 106-106h can output data from the sample after the peritoneal dialysate is removed using the sampling port 134. The step of determining a characteristic of the peritoneal dialysate in operation 312 can include determining the characteristic of the peritoneal dialysate after the peritoneal dialysate is removed using the sampling port 134.

One of ordinary skill in the art will recognize that multiple fluid characteristics can be sampled by sensors 106-106h of systems 100, 200 of FIGS. 1 and 2. Table 2 contains non-limiting examples of sensors 106-106h and corresponding sampled characteristics of the peritoneal dialysate.

TABLE-US-00002 TABLE 2 Sensor Fluid Characteristic Flow sensor Volume of fluid Refractive index sensor Osmotic agent concentration in fluid Conductivity sensor Conductivity of fluid Pressure sensor Pressure to deliver/remove fluid Temperature sensor Temperature of fluid pH sensor pH of fluid Ion selective electrode Concentration of specific ions in fluid

Fluid characteristics of both the peritoneal dialysate in the infusion line being infused into the patient, and the peritoneal dialysate in the effluent line can be determined. Determining the fluid characteristic in both the infusion line and the effluent line allows for determinations of changes to the peritoneal dialysate while inside the peritoneal cavity of the patient during a dwell period. For example, the pH of the peritoneal dialysate infused into the patient and the pH of the peritoneal dialysate removed from the patient allows a determination of the change in pH during the dwell period. A drop in dialysate pH during the dwell period can indicate an infection in the patient, or poor membrane transfer efficiency. Flow sensors in both the infusion line and the effluent line can be used to determine the volume of peritoneal dialysate infused into the patient and the volume of peritoneal dialysate removed from the patient. The difference between the volume of peritoneal dialysate infused into the patient and removed from the patient provides the net fluid removal, or ultrafiltration, from the patient.

The system can divert peritoneal dialysate into the effluent line at multiple times. A portion of the peritoneal dialysate in the peritoneal cavity of the patient can be removed at a first time and a second time, allowing the changes in a fluid characteristic to be determined. A decrease in the pH of the dialysate over time could indicate infection or poor membrane transfer efficiency. Membrane transfer efficiency can also be calculated by measuring changes in solute concentration of the dialysate at multiple times during the dwell period. Concentrations of solutes measured at multiple times during the dwell period can also be used to determine the optimal time to end a peritoneal dialysis cycle. For example, a plateau in the concentration of one or more solutes, including an osmotic agent concentration, could indicate that equilibrium between the patient and the dialysate has been achieved, and a new cycle started.

One skilled in the art will understand that various combinations and/or modifications and variations can be made in the systems and methods depending upon the specific needs for operation. Features illustrated or described as being part of an aspect of the invention can be used in the aspect of the invention, either alone or in combination.

1. A system comprising:
a peritoneal dialysis cycler having an effluent line or a combined infusion and effluent line;
at least one sensor in communication with the effluent line or the combined infusion and effluent line; and
a processor,
wherein the processor is programmed to determine a concentration of at least one solute in an effluent from a patient based on data from the at least sensor.

2. The system of claim 1, wherein the at least one sensor is a refractive index sensor.

3. The system of claim 1, wherein the at least one sensor is an ion selective electrode.

4. The system of claim 1, wherein the at least one sensor is a conductivity sensor.

5. The system of claim 1, wherein the processor is programmed to determine a change in concentration of the at least one solute in a peritoneal dialysate during a dwell time based on data from the at least sensor.

6. The system of claim 1, wherein the processor is programmed to determine a membrane transfer efficiency based on a change in concentration of the at least one solute.

7. The system of claim 1, wherein the processor is programmed to divert peritoneal dialysate from the patient to the at least one sensor at multiple times during a dwell period.

8. The system of claim 1, wherein the at least one sensor is positioned in a sampling flow path fluidly connected to the effluent line or combined infusion and effluent line.

9. The system of claim 1, the system further including:
a second sensor in communication with the effluent line or the combined infusion and effluent line.

10. The system of claim 9, wherein the second sensor is at least one of:
a pH sensor;
a pressure sensor;
a temperature sensor;
a refractive index sensor;
an ion selective electrode;
a conductivity sensor; or
a combination thereof.

11. A method comprising:
sensing, by at least one sensor, a fluid within an effluent line or a combined infusion and effluent line to gather data about the effluent from a patient during peritoneal dialysis therapy, and
determining, by a processor, a concentration of at least one solute in the effluent based on the data from the sensor.

12. The method of claim 11, wherein in the sensing, the at least one sensor is a refractive index sensor.

13. The method of claim 11, wherein in the sensing, the at least one sensor is an ion selective electrode.

14. The method of claim 11, wherein in the sensing, the at least one sensor is a conductivity sensor.

15. The method of claim 11, further comprising:
determining, by the processor, a change in concentration of the at least one solute in a peritoneal dialysate during a dwell time based on the data from the at least one solute concentration sensor.

16. The method of claim 11, further comprising:
determining, by the processor, a membrane transfer efficiency based on a change in concentration of the at least one solute.

17. The method of claim 11, further comprising:
diverting, by the processor, peritoneal dialysate from the patient to the at least one sensor at multiple times during a dwell period.

18. The method of claim 17, further comprising:
determining, by the processor, a change in a fluid characteristic based on the diversion of peritoneal dialysate from the patient to the at least one sensor at multiple times during a dwell period.

19. The method of claim 11, further comprising:
determining, by the processor, an end time for a peritoneal dialysis cycle based on the concentration of the at least one solute.

20. The method of claim 19, further comprising
ending, by the processor, the cycle of the peritoneal dialysis based on the determined end time.
